(19) United States
(12) Patent Application Publication
(10) Pub. No.: US 2020/0000745 A1
(43) Pub. Date: Jan. 2, 2020

(54) Chemical medicinal composition used for the improvement of sleep

(71) Applicant: Kimberly Denise Wesley, Mountain House, CA (US)

(72) Inventor: Kimberly Denise Wesley, Mountain House, CA (US)

(21) Appl. No.: 15/972,192

(22) Filed: Jul. 2, 2018

(51) Int. Cl. A61K 31/133; A61P 25/20

(52) U.S. Cl. CPC A61K 31/133; A61P 25/20

(57) ABSTRACT

The compounds are of the class of antioxidant biogenic amines and a class of ethanolamines that are used to treat short-term insomnia by promoting sleep onset and maintenance.

A medicinal preparation to alleviate sleeplessness using the molecular formula;

C₃₀H₃₈CINO₃

The chemical compound used for sleeplessness does not require a physicians prescription and is non-habit forming.

A method for treating insomnia characterized by difficulties with sleep maintenance, the method comprises administering a dosage of the chemical medicinal composition of C₃₀H₃₈CINO₃ or a pharmaceutically acceptable salt thereof to a patient having a sleep disorder in which, for a given eight-hour period of desired sleep, the patient experiences interrupted sleep during the first 60 minutes of initial sleep. the desired dose is between 6 mg and 15 mg of the chemical or medicinal composition of C₃₀H₃₈CINO₃ or a pharmaceutically acceptable salt given prior to the start of sleep.

1. A method for treating insomnia characterized by difficulties with sleep maintenance, wherein the method comprises administering a dosage of the chemical medicinal composition of C₃₀H₃₈CINO₃ or a pharmaceutically acceptable salt thereof to a patient having a sleep disorder in which, for a given eight-hour period of desired sleep, the patient experiences interrupted sleep during the first 60 minutes of initial sleep. the desired dose is between 6 mg and 15 mg of the chemical or medicinal composition of C₃₀H₃₈CINO₃ or a pharmaceutically acceptable salt given prior to the start of sleep.
